(19) United States
(12) Patent Application Publication
(10) Pub. No.: US 2012/0157477 A1
(43) Pub. Date: Jun. 21, 2012

(54) REVERSAL OF L-DOPA-INDUCED DYSKINESIA BY NEURONAL NICOTINIC RECEPTOR LIGANDS

(75) Inventors: Kristen G. Jordan, Clemmons, NC (US); Sharon Rae Letchworth, Kernersville, NC (US); Merouane Bencherif, Winston-Salem, NC (US)

(73) Assignee: TARGACEPT, INC., Winston-Salem, NC (US)

(21) Appl. No.: 13/378,089

(22) PCT Filed: Jun. 15, 2010
(86) PCT No.: PCT/US10/38605

§ 371 Date: Mar. 8, 2012

Related U.S. Application Data
(60) Provisional application No. 61/187,896, filed on Jun. 17, 2009.

(51) Int. Cl. A61K 31/506; A61P 25/14; A61P 25/16; A61P 1/06

(52) U.S. Cl. 514/256

(57) ABSTRACT

The present invention includes methods, uses, and compounds for treating or preventing L-dopa-induced dyskinesias.

### BACKGROUND

Dyskinesia, or abnormal involuntary movements (AIMs), develop in the majority of Parkinson's disease (PD) patients with long-term L-dopa use and may be as debilitating as PD itself. Quik and coworkers recently showed that nicotine reduces L-dopa-induced dyskinetic-like movements by ˜50% in both Parkinsonian nonhuman primates and rodents. Available treatments for L-dopa-induced AIMs are currently very limited. Therefore, the observation that nicotine treatment reduces these movements may provide a novel therapeutic option, particularly because nicotine does not compromise the anti-Parkinsonian action of L-dopa.

Nicotine generally exerts its effect by stimulating neuronal nicotinic receptors (NNRs). However, nicotine targets multiple NNRs in the central and peripheral nervous system and in skeletal muscle. This leads to side effects, in addition to any desired response. Thus, it would be advantageous to identify NNR subtype-selective drugs that reduce L-dopa-induced dyskinesias.

Reference is made to the following citations: Carta, M., Carlsson, T., Munoz, A., Kirik, D. & Bjorklund, A., “Serotonin-Dopamine Interaction In The Induction And Maintenance Of L-Dopa-Induced Dyskinesias,” Prog Brain Res, 172: 465-78 (2008); Cenci, M. A. & Lundblad, M., “Ratings Of L-Dopa-Induced Dyskinesia In The Unilateral 6-OHDA Lesion Model Of Parkinson's Disease In Rats And Mice,” Curr Protoc Neurosci, Chapter 9, Unit 9: 25 (2007); Cox, H. et al., “The Selective Kappa-Opioid Receptor Agonist U50,488 Reduces L-Dopa-Induced Dyskinesias But Worsens Parkinsonism In MPTP-Treated Primates,” Exp Neurol, (2007); Dani, J. A. & Bertrand, D., “Nicotinic Acetylcholine Receptors And Nicotinic Cholinergic Mechanisms Of The Central Nervous System,” Annu Rev Pharmacol Toxicol, 47: 699-729 (2007); Dekundy, A., Lundblad, M., Danysz, W. & Cenci, M. A, “Modulation Of L-Dopa-Induced Abnormal Involuntary Movements By Clinically Tested Compounds: Further Validation Of The Rat Dyskinesia Model,” Behav Brain Res, 179: 76-89 (2007); Exley, R. & Cragg, S. J., “Presynaptic Nicotinic Receptors: A Dynamic And Diverse Cholinergic Filter Of Striatal Dopamine Neurotransmission,” Br J Pharmacol, 153 Suppl 1: S283-97 (2008); Fox, S. H., Lang, A. E. & Brotchie, J. M., “Translation Of Nondopaminergic Treatments For Levodopa-Induced Dyskinesia From MPTP-Lesioned Nonhuman Primates To Phase IIA Clinical Studies Keys To Success And Roads To Failure,” Mov Disord, 21, 1578-94 (2006); Gotti, C. et al., “Heterogeneity And Complexity Of Native Brain Nicotinic Receptors,” Biochem Pharmacol,” 74: 1102-11 (2007); Grady, S. R. et al., “The Subtypes Of Nicotinic Acetylcholine Receptors On Dopaminergic Terminals Of Mouse Striatum,” Biochem Pharmacol, 74: 1235-46 (2007); Guigoni, C., et al., “Pathogenesis Of Levodopa-Induced Dyskinesia: Focus On D1 And D3 Dopamine Receptors,” Parkinsonism Relat Disord, 11 Suppl 1: S25-9 (2005); Guigoni, C. et al., “Involvement Of Sensorimotor, Limbic, and Associative Basal Ganglia Domains In L-3,4-Dihydroxyphenylalanine-Induced Dyskinesia,” J Neurosci, 25: 2102-7 (2005); Hsu, A. et al., “Effect Of The D3 Dopamine Receptor Partial Agonist BP-897 [N-[4-(4-(2-Methoxyphenyl)piperazinyl)Butyl]-2-Naphthamide] On L-3,4-Dihydroxyphenylalanine-Induced Dyskinesias And Parkinsonism In Squirrel Monkeys,” J Pharmacol Exp Ther, 311: 770-7 (2004); Linazasoro, G., “New Ideas On The Origin Of L-Dopa-Induced Dyskinesias: Age, Genes And Neural Plasticity,” Trends Pharmacol Sci, 26: 391-7 (2005); Linazasoro, G., Van Blercom, N., Ugedo, L. & Ruiz Ortega, J. A., “Pharmacological Treatment Of Parkinson's Disease: Life Beyond Dopamine D2/D3 Receptors?” J Neural Transm, 115: 431-41 (2008); Mercuri, N. B. & Bernardi, G., “The ‘Magic’ Of L-Dopa: Why Is It The Gold Standard Parkinson's Disease Therapy?” Trends Pharmacol Sci, 26: 341-4 (2005); Quik, M., Police, S., He, L., Di Monte, D. A. & Langston, J. W., “Expression Of D(3) Receptor Messenger RNA And Binding Sites In Monkey Striatum And Substantia Nigra After Nigrostriatal Degeneration: Effect Of Levodopa Treatment,” Neuroscience, 98: 263-73 (2000); Quik, M., Police, S., Langston, J. W. & Di Monte, D. A., “Increases In Striatal Preproenkephalin Gene Expression Are Associated With Nigrostriatal Damage But Not L-Dopa-Induced Dyskinesias In The Squirrel Monkey,” Neuroscience, 113: 213-20 (2002); Quik, M., Polonskaya, Y., Mcintosh, J. M. & Kulak, J. M., “Differential Nicotinic Receptor Expression In Monkey Basal Ganglia: Effects Of Nigrostriatal Damage,” Neuroscience, 112: 619-30 (2002); Quik, M. et al., “L-Dopa Treatment Modulates Nicotinic Receptors In Monkey Striatum,” Mol Pharmacol, 64: 619-28 (2003); Quik, M. et al., “Chronic Oral Nicotine Treatment Protects Against Striatal Degeneration In MPTP-Treated Primates,” J Neurochem, 98: 1866-75 (2006); Quik, M., O'leary, K. & Tanner, C. M., “Nicotine and Parkinson's Disease: Implications For Therapy,” Mov Disord, 23: 1641-52 (2008), Samadi, P., Bedard, P. J. & Rouillard, C., “Opioids And Motor Complications In Parkinson's Disease,” Trends Pharmacol Sci, 27: 512-7 (2006); Togasaki, D. M. et al., “Levodopa Induces Dyskinesias In Normal Squirrel Monkeys,”. Ann Neurol, 50: 254-7. (2001).

### SUMMARY OF THE INVENTION

One aspect of the present invention includes a method for treating L-dopa-induced dyskinesia or abnormal involuntary movements by administering (R)-5-((E)-2-pyrrolidin-3-ylvinyl)pyrimidine or a pharmaceutically acceptable salt thereof. Another aspect includes a method for reducing L-dopa-induced dyskinesia or abnormal involuntary movements by administering (R)-5-((E)-2-pyrrolidin-3-ylvinyl)pyrimidine or a pharmaceutically acceptable salt thereof. Another aspect includes a method for delaying the onset or progression of L-dopa-induced dyskinesia or abnormal involuntary movements by administering (R)-5-((E)-2-pyrrolidin-3-ylvinyl)pyrimidine or a pharmaceutically acceptable salt thereof. Another aspect includes a method for improving Parkinsonism by administering (R)-5-((E)-2-pyrrolidin-3-ylvinyl)pyrimidine or a pharmaceutically acceptable salt thereof. Another aspect includes a method for treating a disease responsive to L-dopa with L-dopa and (R)-5-((E)-2-pyrrolidin-3-ylvinyl)pyrimidine or a pharmaceutically acceptable salt thereof. In one embodiment, the disease responsive to L-dopa is Parkinson's disease. Another aspect includes a method for treating, reducing, or delaying progression of L-dopa-induced dyskinesia or abnormal involuntary movements by administering a compound which targets both α4β2* and α6β2* NNRs.

Similarly, one aspect of the present invention includes use of (R)-5-((E)-2-pyrrolidin-3-ylvinyl)pyrimidine or a pharmaceutically acceptable salt thereof in the manufacture of a medicament for treating L-dopa-induced dyskinesia or abnormal involuntary movements. Another aspect includes use of (R)-5-((E)-2-pyrrolidin-3-ylvinyl)pyrimidine or a pharmaceutically acceptable salt thereof in the manufacture of a medicament for reducing L-dopa-induced dyskinesia or abnormal involuntary movements. Another aspect includes use of (R)-5-((E)-2-pyrrolidin-3-ylvinyl)pyrimidine or a pharmaceutically acceptable salt thereof in the manufacture of a medicament for delaying the onset or progression of L-dopa-induced dyskinesia or abnormal involuntary movements. Another aspect includes use of (R)-5-((E)-2-pyrrolidin-3-ylvinyl)pyrimidine or a pharmaceutically acceptable salt thereof in the manufacture of a medicament for improving Parkinsonism. Another aspect includes use of each of L-dopa and (R)-5-((E)-2-pyrrolidin-3-ylvinyl)pyrimidine or a pharmaceutically acceptable salt thereof in the manufacture of a medicament for treating a disease responsive to L-dopa. In one embodiment, the disease responsive to L-dopa is Parkinson's disease. Another aspect includes use of a compound which targets both α4β2* and α6β2* NNRs in the manufacture of a medicament for treating, reducing, or delaying progression of L-dopa-induced dyskinesia or abnormal involuntary movements.

Similarly, one aspect of the present invention includes a compound, (R)-5-((E)-2-pyrrolidin-3-ylvinyl)pyrimidine or a pharmaceutically acceptable salt thereof, for treating L-dopa-induced dyskinesia or abnormal involuntary movements. Another aspect includes a compound, (R)-5-((E)-2-pyrrolidin-3-ylvinyl)pyrimidine or a pharmaceutically acceptable salt thereof, for reducing L-dopa-induced dyskinesia or abnormal involuntary movements. Another aspect includes a compound, (R)-5-((E)-2-pyrrolidin-3-ylvinyl)pyrimidine or a pharmaceutically acceptable salt thereof, for delaying the onset or progression of L-dopa-induced dyskinesia or abnormal involuntary movements. Another aspect includes a compound, (R)-5-((E)-2-pyrrolidin-3-ylvinyl)pyrimidine or a pharmaceutically acceptable salt thereof, for improving Parkinsonism. Another aspect includes a compound, (R)-5-((E)-2-pyrrolidin-3-ylvinyl)pyrimidine or a pharmaceutically acceptable salt thereof, in combination with L-dopa for treating a disease responsive to L-dopa. In one embodiment, the disease responsive to L-dopa is Parkinson's disease. Another aspect includes a compound, which targets both α4β2* and α6β2* NNRs, for treating, reducing, or delaying progression of L-dopa-induced dyskinesia or abnormal involuntary movements.

Another aspect of the present invention includes a kit comprising: L-dopa; (R)-5-((E)-2-pyrrolidin-3-ylvinyl)pyrimidine or a pharmaceutically acceptable salt thereof; and one or more instruction regarding a treatment regimen to treat, reduce, or delay onset or progression of L-dopa-induced dyskinesia or abnormal involuntary movements. In one embodiment, such a kit may also include packaging, such as a blister pack. Alternatively, such a kit may provide for individual prescription and dosing of each of the L-dopa and (R)-5-((E)-2-pyrrolidin-3-ylvinyl)pyrimidine or a pharmaceutically acceptable salt thereof, but when combined with the instruction regarding a treatment regimen to treat, reduce, or delay onset or progression of L-dopa-induced dyskinesia or abnormal involuntary movements, such is intended to be within the scope of the present invention.

Another aspect includes a pharmaceutical composition comprising: L-dopa; (R)-5-((E)-2-pyrrolidin-3-ylvinyl)pyrimidine or a pharmaceutically acceptable salt thereof; and one or more pharmaceutically acceptable carrier. In one embodiment, the pharmaceutical composition may be a unitary dosage form.

Another aspect includes a combination comprising: L-dopa; and (R)-5-((E)-2-pyrrolidin-3-ylvinyl)pyrimidine or a pharmaceutically acceptable salt thereof. In one embodiment, the combination may occur as separate dosage forms with each active ingredient, administered together or separate, sequentially or concurrently, and close in time or remote in time to each other.

Another aspect includes a treatment regimen comprising: L-dopa; and (R)-5-((E)-2-pyrrolidin-3-ylvinyl)pyrimidine or a pharmaceutically acceptable salt thereof, wherein the (R)-5-((E)-2-pyrrolidin-3-ylvinyl)pyrimidine or a pharmaceutically acceptable salt thereof, is administered concurrently with the L-dopa.

The present invention includes combinations of aspects and embodiments, as well as preferences, as herein described throughout the present specification.

### BRIEF DESCRIPTION OF THE FIGURES

The Figures describe results obtained according to particular embodiments of the invention and exemplify aspects of the invention but should not be construed to be limiting.

FIG. 1 is a rendering of the treatment schedule for the L-dopa-induced dyskinesia study in rats. FIG. 1 depicts the time, Compound A (which is also referred to as Agonist #PD1) treatment, L-dopa dosing, and behavioral testing.

FIG. 2 is a graphic representation showing that two separate doses of Compound A (again, which is also referred to as PD1) reduce L-dopa-induced AIMs over the course of several assessments in Parkinsonian rats. Each value is the mean±SEM of 10 rats. Significance of difference from control; P<0.05.

FIG. 3 is a graphic representation showing that two separate doses of Compound A (again, which is also referred to as PD1) do not worsen parkinsonism Off (in the absence of) or On (in the presence of) L-dopa as assessed using the asymmetric forelimb or cylinder test on several occasions during the course of the study. Each value is the mean±SEM of 9-10 rats.

### DETAILED DESCRIPTION OF THE PREFERRED EMBODIMENTS

There is a well-recognized problem with the development of dyskinesias with prolonged use of L-dopa treatment for movement disorders, including but not limited to Parkinson's disease. Dyskinesia is an acknowledged and well-characterized clinical side effect of L-dopa treatment. See, for example, Brotchie, J. M., Lee, J. & Venderova, K., “Levodopa-Induced Dyskinesia in Parkinson's Disease,” J Neural Transm, 112: 359-91 (2005); Fabbrini, G., Brotchie, J. M., Grandas, F., Nomoto, M. & Goetz, C. G., “Levodopa-Induced Dyskinesias,” Mov Disord (2007); Olanow, C. W., Obeso, J. A. & Stocchi, F., “Continuous Dopamine-Receptor Treatment of Parkinson's Disease: Scientific Rationale and Clinical Implications,” Lancet Neurol, 5: 677-87 (2006); Stacy, M. & Galbreath, A., “Optimizing Long-Term Therapy For Parkinson Disease: Options For Treatment-Associated Dyskinesia,”. Clin Neuropharmacol, 31: 120-5 (2008); Stacy, M. & Galbreath, A., “Optimizing Long-Term Therapy For Parkinson Disease: Levodopa, Dopamine Agonists, And Treatment-Associated Dyskinesia,” Clin Neuropharmacol, 31: 51-6 (2008); and Thanvi, B., Lo, N. & Robinson, T., “Levodopa-Induced Dyskinesia In Parkinson's Disease: Clinical Features, Pathogenesis, Prevention And Treatment,” Postgrad Med J, 83: 384-8 (2007).

In 2007, published studies demonstrated specific relevance of the prototypical nicotinic acetylcholine agonist nicotine for use in amelioration of L-dopa-induced dyskinesias in animal models of Parkinson's disease. See, Bordia, T., Campos, C., Huang, L. Z. & Quik, M., “Continuous And Intermittent Nicotine Treatment Reduces L-3,4-Dihydroxyphenylalanine (L-DOPA)-Induced Dyskinesias In A Rat Model Of Parkinson's Disease,” J Pharmacol Exp Ther, 327: 239-47 (2008); Quik, M. et al., “Nicotine Reduces Levodopa-Induced Dyskinesias In Lesioned Monkeys,” Annals of Neurology, 62: 588-96 (2007); and Quik, M., Bordia, T. & Weary, K., “Nicotinic Receptors As CNS Targets For Parkinson's Disease,”. Biochem Pharmacol, 74: 1224-1234 (2007).

Although the data were quite compelling, a number of undesirable side effects are also correlated with nicotine use and administration in humans, specifically related to its effects in the periphery, namely muscle and ganglion. Therefore alternative compounds with more selective neuronal nicotinic receptor interactions are believed to provide an advantage with respect to patient compliance and therapeutic index.

The primary NNRs in the striatum, but not in the peripheral nervous system, are the α4β2*, α6β2* and α7 NNRs, where the asterisk denotes the possible presence of other NNR subunits in the receptor complex. Therefore, drugs directed to these NNR subtypes should provide an improved alternative to amelioration to L-dopa-induced dyskinesias over nicotine.

The following definitions are meant to refine but not limit, the terms defined. If a particular term used herein is not specifically defined, such term should not be considered indefinite. Rather, terms are used within their accepted meanings.

As used herein, an “agonist” is a substance that stimulates its binding partner, typically a receptor. Stimulation is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Stimulation may be defined with respect to an increase in a particular effect or function that is induced by interaction of the agonist or partial agonist with a binding partner and can include allosteric effects.

As used herein, an “antagonist” is a substance that inhibits its binding partner, typically a receptor. Inhibition is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Inhibition may be defined with respect to a decrease in a particular effect or function that is induced by interaction of the antagonist with a binding partner, and can include allosteric effects.

As used herein, a “partial agonist” or a “partial antagonist” is a substance that provides a level of stimulation or inhibition, respectively, to its binding partner that is not fully or completely agonistic or antagonistic, respectively. It will be recognized that stimulation, and hence, inhibition is defined intrinsically for any substance or category of substances to be defined as agonists, antagonists, or partial agonists.

As used herein, “intrinsic activity” or “efficacy” relates to some measure of biological effectiveness of the binding partner complex. With regard to receptor pharmacology, the context in which intrinsic activity or efficacy should be defined will depend on the context of the binding partner (e.g., receptor/ligand) complex and the consideration of an activity relevant to a particular biological outcome. For example, in some circumstances, intrinsic activity may vary depending on the particular second messenger system involved. See Hoyer, D. and Boddeke, H., Trends Pharmacol. Sci. 14(7): 270-5 (1993), herein incorporated by reference with regard to such teaching. Where such contextually specific evaluations are relevant, and how they might be relevant in the context of the present invention, will be apparent to one of ordinary skill in the art.

As used herein, modulation of a receptor includes agonism, partial agonism, antagonism, partial antagonism, or inverse agonism of a receptor.

As used throughout this specification, Compound A (also referred to as PD1 in biological testing) is (R)-5-((E)-2-pyrrolidin-3-ylvinyl)pyrimidine or a pharmaceutically acceptable salt thereof, illustrated below. Compound A has the following structural formula:

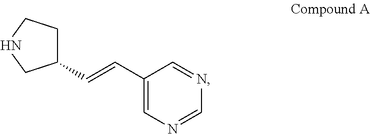

or a pharmaceutically acceptable salt thereof. (R)-5-((E)-2-pyrrolidin-3-ylvinyl)pyrimidine, its racemic synthesis, and its galatarate and hemi-galactarate salt forms are disclosed in published WO 04/078752 and U.S. Pat. No. 7,098,331, each of which is incorporated by reference herein for their disclosure of the preparation of Compound A.

A study to evaluate the effect of NNR drugs that target CNS subtypes in a well-validated Parkinsonian animal model of dyskinesias (L-dopa-treated unilateral 6-hydroxydopamine-lesioned rats) was initiated. Compound A (0.1 or 0.3 mg/kg/d via subcutaneous minipump), which targets both α4β2* and α6β2* NNRs, reduced total L-dopa-induced AIMS by ˜25% with a more pronounced reduction in oral than axial AIMs.

In addition, Compound A demonstrated a trend for improving Parkinsonism in lesioned rats. Thus, selective NNR drugs may be useful for the management of dyskinesias in Parkinson's disease, a problematic complication of L-dopa therapy for which there is currently very limited treatment.

As used herein, the term “pharmaceutically acceptable” refers to carrier(s), diluent(s), excipient(s) or salt forms of the compounds of the present invention that are compatible with the other ingredients of the formulation and not deleterious to the recipient of the pharmaceutical composition.

As used herein, the term “pharmaceutical composition” refers to a compound of the present invention optionally admixed with one or more pharmaceutically acceptable carriers, diluents, or excipients. Pharmaceutical compositions preferably exhibit a degree of stability to environmental conditions so as to make them suitable for manufacturing and commercialization purposes.

As used herein, the terms “effective amount”, “therapeutic amount”, and “effective dose” refer to an amount of the compound of the present invention sufficient to elicit the desired pharmacological or therapeutic effects, thus resulting in an effective treatment of a disorder. Treatment of a disorder may be manifested by delaying or preventing the onset or progression of the disorder, as well as the onset or progression of symptoms associated with the disorder. Treatment of a disorder may also be manifested by a decrease or elimination of symptoms, reversal of the progression of the disorder, as well as any other contribution to the well being of the patient.

The effective dose can vary, depending upon factors such as the condition of the patient, the severity of the symptoms of the disorder, and the manner in which the pharmaceutical composition is administered. To be administered in an effective dose, compounds may be administered in an amount of as low as 0.1 mg/kg of patient weight. In one embodiment, the compound may be administered in an amount from less than about 1 mg/kg patient weight to less than about 100 μg/kg of patient weight, and further between about 1 μg/kg to less than 100 μg/kg of patient weight. The foregoing effective doses typically represent that amount that may be administered as a single dose, or as one or more doses that may be administered over a 24 hours period. In one embodiment, a daily dose of Compound A may be administered of about 1 mg to about 100 mg, preferably 1 mg to about 50 mg, preferably about 5 mg to about 30 mg, preferably about 5 mg to about 20 mg. The dose may be once daily or may be divided so as to provide twice daily (BID), three times a day (QD), four times a day (QID), or more doses.

Compound A or a pharmaceutically acceptable salt thereof may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Compound A contains a chiral center and, therefore may be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be made by any suitable method known in the art. See, for example, Stereochemistry of Organic Compounds (Wiley-Interscience, 1994).

The present invention includes a salt or solvate of the compounds herein described, including combinations thereof such as a solvate of a salt. The compounds of the present invention may exist in solvated, for example hydrated, as well as unsolvated forms, and the present invention encompasses all such forms. Typically, but not absolutely, the salts of the present invention are pharmaceutically acceptable salts. Salts encompassed within the term “pharmaceutically acceptable salts” refer to non-toxic salts of the compounds of this invention. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

Although it is possible to administer the compound of the present invention in the form of a bulk active chemical, it is preferred to administer the compound in the form of a pharmaceutical composition or formulation. Thus, one aspect the present invention includes pharmaceutical compositions comprising one or more compounds of Formula I and/or pharmaceutically acceptable salts thereof and one or more pharmaceutically acceptable carriers, diluents, or excipients. Another aspect of the invention provides a process for the preparation of a pharmaceutical composition including admixing one or more compounds of Formula I and/or pharmaceutically acceptable salts thereof with one or more pharmaceutically acceptable carriers, diluents or excipients.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions can also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline.

The formulations may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses.

The administration of the pharmaceutical compositions described herein can be intermittent, or at a gradual, continuous, constant or controlled rate. The pharmaceutical compositions may be administered to a warm-blooded animal, for example, a mammal such as a mouse, rat, cat, rabbit, dog, pig, cow, or monkey; but advantageously is administered to a human being. In addition, the time of day and the number of times per day that the pharmaceutical composition is administered can vary.

Compound A or a pharmaceutically acceptable salt thereof may be used in combination with a variety of other suitable therapeutic agents useful in the treatment or prophylaxis of those disorders or conditions. Thus, one embodiment of the present invention includes the administration of the compound of the present invention in combination with other therapeutic compounds. For example, the compound of the present invention can be used in combination with other NNR ligands (such as varenicline), allosteric modulators of NNRs, antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time. Another aspect of the present invention includes combination therapy comprising administering to the subject a therapeutically or prophylactically effective amount of the compound of the present invention and one or more other therapy including chemotherapy, radiation therapy, gene therapy, or immunotherapy.

The compounds of the present invention, when employed in effective amounts, are believed to modulate the activity of the α4β2 and α6β2*, with little to no effect at α7 without appreciable interaction with the nicotinic subtypes that characterize the human ganglia, as demonstrated by a lack of the ability to elicit nicotinic function in adrenal chromaffin tissue, or skeletal muscle, further demonstrated by a lack of the ability to elicit nicotinic function in cell preparations expressing muscle-type nicotinic receptors. Thus, these compounds are believed capable of treating or preventing diseases, disorders and conditions without eliciting significant side effects associated activity at ganglionic and neuromuscular sites. Thus, administration of the compounds is believed to provide a therapeutic window in which treatment of certain diseases, disorders and conditions is provided, and certain side effects are avoided. That is, an effective dose of the compound is believed sufficient to provide the desired effects upon the disease, disorder or condition, but is believed insufficient, namely is not at a high enough level, to provide undesirable side effects.

The compounds can be used in diagnostic compositions, such as probes, particularly when they are modified to include appropriate labels. For this purpose the compounds of the present invention most preferably are labeled with a radioactive isotopic moiety such as ¹¹C, ¹⁸F, ⁷⁶Br, ¹²³I or ¹²⁵I. The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al., as noted. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al., each herein incorporated by reference, for a disclosure of representative imaging techniques.

In one aspect, the diagnostic compositions can be used in a method to diagnose disease in a subject, such as a human patient. The method involves administering to that patient a detectably labeled compound as described herein, and detecting the steric mechanism of that compound to selected NNR subtypes (e.g., α4β2 and α6β2* receptor subtypes). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that can be evaluated include those that are set forth in U.S. Pat. No. 5,952,339 to Bencherif et al.

In another aspect, the diagnostic compositions can be used in a method to monitor selective nicotinic receptor subtypes of a subject, such as a human patient. The method involves administering a detectably labeled compound as described herein to that patient and detecting the ion flux impact of that compound to selected nicotinic receptor subtypes namely, the α4β2 and α6β2* receptor subtypes.

Compound A may be made by a variety of methods, including well-established synthetic methods. Illustrative general synthetic methods are set out below and then specific compounds of the invention are prepared in the working Examples.

In the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons, herein incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

Compound A can be prepared according to the methods described below using readily available starting materials and reagents. In these reactions, variants may be employed which are themselves known to those of ordinary skill in this art but are not described in detail here.

Unless otherwise stated, structures depicted herein are also meant to include compounds which differ only in the presence of one or more isotopically enriched atoms. Compounds having the present structure except for the replacement of a hydrogen atom by a deuterium or tritium, or the replacement of a carbon atom by a ¹³C— or ¹⁴C-enriched carbon are within the scope of the invention. For example, deuterium has been widely used to examine the pharmacokinetics and metabolism of biologically active compounds. Although deuterium behaves similarly to hydrogen from a chemical perspective, there are significant differences in bond energies and bond lengths between a deuterium-carbon bond and a hydrogen-carbon bond. Consequently, replacement of hydrogen by deuterium in a biologically active compound may result in a compound that generally retains its biochemical potency and selectivity but manifests significantly different absorption, distribution, metabolism, and/or excretion (ADME) properties compared to its isotope-free counterpart. Thus, deuterium substitution may result in improved drug efficacy, safety, and/or tolerability for some biologically active compounds.

Those skilled in the art of organic synthesis will appreciate that there exist multiple means of producing compounds of the present invention, as well as means for producing compounds of the present invention which are labeled with a radioisotope appropriate to various uses. For example, coupling with a ¹¹C- or ¹⁸F-labeled aryl- or heteroarylboronic acid, followed by removal of the protecting group, as described above will produce a compound suitable for use in positron emission tomography. Likewise, coupling with a ³H- or ¹⁴C-labeled aryl- or heteroarylboronic acid, followed by removal of the protecting group, as described above will produce an isotopically modified compound suitable for use in functional and metabolism studies or as an alternative therapeutic compound.

### EXAMPLES

### Example 1

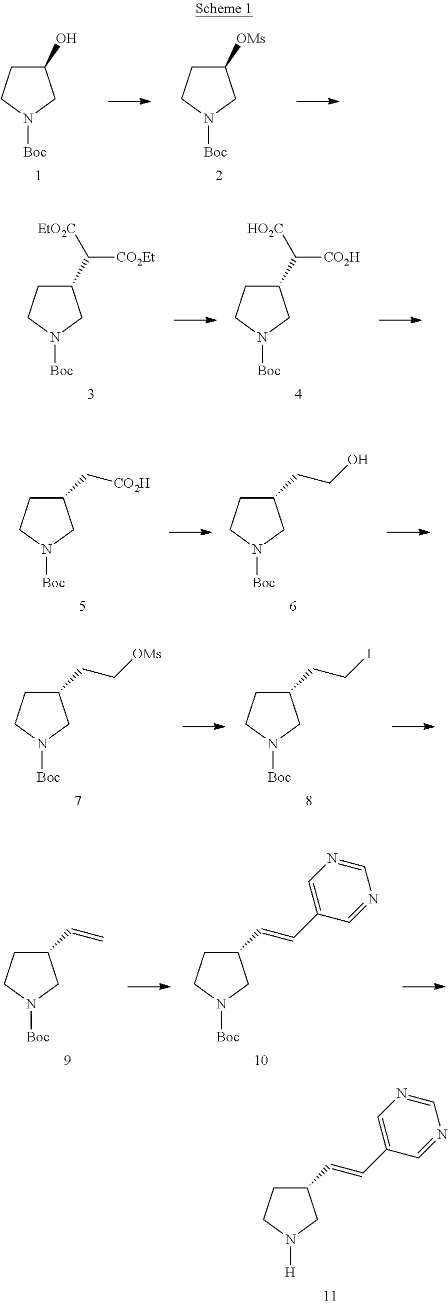

### Synthesis of tert-butyl (R)-3-(methylsulfonyloxy)pyrrolidine-1-carboxylate (2)

Procedure A: To a solution of tert-butyl (R)-3-hydroxypyrrolidine-1-carboxylate (200 g, 1.07 mol) and triethylamine (167 g, 1.63 mol) in toluene (700 mL) at −20 to −30° C. was added methanesulfonyl chloride (156 g, 1.36 mol) drop-wise while maintaining the temperature at −10 to −20° C. The solution was warmed to ambient temperature and allowed to stir. The reaction solution was sampled hourly and analyzed by HPLC to establish completion of the reaction. Upon completion of the reaction, the suspension was filtered to remove the triethylamine hydrochloride. The filtrate was washed with ˜600 mL of dilute aqueous sodium bicarbonate solution. The organic layer was dried and concentrated under reduced pressure to give 2 as a viscous oil (260 g, 92%) which is used without further purification. ¹H NMR (CDCl₃, 400 MHz) δ 5.27 (m, 1H), 3.44-3.76 (m, 4H), 3.05 (s, 3H), 2.26 (m, 1H), 2.15 (m, 1H), 1.47 (s, 9H).

Procedure B: A reactor was charged with tert-butyl (R)-3-hydroxypyrrolidine-1-carboxylate (2.00 kg, 10.7 mol), toluene (8.70 kg) and triethylamine (1.75 kg, 17.3 mol). The reactor was flushed with nitrogen for 15 min. The mixture was stirred and cooled to 3° C. Methanesulfonyl chloride (1.72 kg, mol) was slowly added (over a 2 h period) with continuous ice bath cooling (exothermic reaction) (after complete addition, the temperature was 14° C.). The mixture, now viscous with precipitated triethylamine hydrochloride, was stirred 12 h as it warmed to 20° C. Both GC and TLC analysis (ninhydrin stain) indicated that no starting material remained. The mixture was filtered to remove the triethylamine hydrochloride, and the filtrate was returned to the reactor. The filtrate was then washed (2×3 kg) with 5% aqueous sodium bicarbonate, using 15 min of stirring and 15 min of settling time for each wash. The resulting organic layer was dried over anhydrous sodium sulfate and filtered. The volatiles were removed from the filtrate under vacuum, first at 50° C. for 4 h and then at ambient temperature for 10 h. The residue weighed 3.00 kg (106% yield) and was identical by chromatographic and NMR analysis to previously prepared samples, with the exception that it contained toluene.

### Synthesis of diethyl (R)-2-(1-(tert-butoxycarbonyl)pyrrolidin-3-yl)malonate (3)

Preparation A: To a solution of potassium tert-butoxide (187 g, 1.62 mol) in 1-methyl-2-pyrrolidinone (1.19 L) was added diethyl malonate (268 g. 1.67 mol) while maintaining the temperature below 35° C. The solution was heated to 40° C. and stirred for 20-30 min. tert-Butyl (R)-3-(methylsulfonyloxyl)pyrrolidine-1-carboxylate (112 g, 420 mmol) was added and the solution was heated to 65° C. and stirred for 6 h. The reaction solution was sampled every 2 h and analyzed by HPLC to establish completion of the reaction. Upon completion of reaction (10-12 h), the mixture was cooled to around 25° C. De-ionized water (250 mL) was added to the solution, and the pH was adjusted to 3-4 by addition of 2N hydrochloric acid (650 mL). The resulting suspension was filtered, and water (1.2 L) and chloroform (1.4 L) were added. The solution was mixed thoroughly, and the chloroform layer was collected and evaporated under reduced pressure to give a yellow oil. The oil was dissolved in hexanes (2.00 L) and washed with deionized water (2×1.00 L). The organic layer was concentrated under reduced pressure at 50-55° C. to give a pale yellow oil (252 g) which ¹H NMR analysis indicates to be 49.1% of 3 (123.8 g) along with 48.5% diethyl malonate (122 g), and 2% of 1-methyl-2-pyrrolidinone (5 g). The material was carried forward to the next step without further purification. ¹H NMR (CDCl₃, 400 MHz) δ 4.20 (q, 4H), 3.63 (m, 1H), 3.48 (m, 1H), 3.30 (m, 1H), 3.27 (d, J=10 Hz, 1H), 3.03 (m, 1H), 2.80 (m, 1H), 2.08 (m, 1H), 1.61 (m, 1H), 1.45 (s, 9H), 1.27 (t, 6H).

Preparation B: A reactor, maintained under a nitrogen atmosphere, was charged with 200 proof ethanol (5.50 kg) and 21% (by weight) sodium ethoxide in ethanol (7.00 kg, 21.6 mol). The mixture was stirred and warmed to 30° C. Diethyl malonate (3.50 kg, 21.9 mol) was added over a 20 min period. The reaction mixture was then warmed at 40° C. for 1.5 h. A solution of tert-butyl (R)-3-(methylsulfonyloxyl)pyrrolidine-1-carboxylate (3.00 kg of the product from Example 2, Procedure B, 10.7 mol) in 200 proof ethanol (5.50 kg) was added, and the resulting mixture was heated at reflux (78° C.) for 2 h. Both GC and TLC analysis (ninhydrin stain) indicated that no starting material remained. The stirred mixture was then cooled to 25° C., diluted with water (2.25 kg), and treated slowly with a solution of concentrated hydrochloric acid (1.27 kg, 12.9 mol) in water (5.44 kg). This mixture was washed twice with methyl tert-butyl ether (MTBE) (14.1 kg and 11.4 kg), using 15 min of stirring and 15 min of settling time for each wash. The combined MTBE washes were dried over anhydrous sodium sulfate (1 kg), filtered and concentrated under vacuum at 50° C. for 6 h. The residue (red oil) weighed 4.45 kg and was 49% desired product by GC analysis (62% overall yield from tert-butyl (R)-3-hydroxypyrrolidine-1-carboxylate).

### Synthesis of (R)-2-(1-(tert-butoxycarbonyl)pyrrolidin-3-yl)malonic acid (4)

Procedure A: To a solution of the product of Example 3, Procedure A (232 g), containing 123.8 g (380 mmol) of 3 and 121.8 g (760 mmol) of diethyl malonate, in tetrahydrofuran (1.2 L) was added a 21% potassium hydroxide solution (450 g in 0.50 L of deionized water) while maintaining the temperature below 25° C. The reaction mixture was heated to 45° C. and stirred for 1 h. The reaction solution was sampled every hour and analyzed by HPLC to establish completion of the reaction. Upon completion of reaction (2-3 h), the mixture was cooled to around 25° C. The aqueous layer was collected and cooled to 5° C. The pH was adjusted to 2 by addition of 4N hydrochloric acid (750 mL), and the resulting suspension was held at 5-10° C. for 30 min. The mixture was filtered, and the filter cake was washed with hexanes (1 L). The aqueous filtrate was extracted with chloroform (1 L) and the chloroform layer was put aside. The solids collected in the filtration step were re-dissolved in chloroform (1 L) by heating to 40° C. The solution was filtered to remove un-dissolved inorganic solids. The chloroform layers were combined and concentrated under reduced pressure at 50-55° C. to give an off-white solid (15 g). The solids were combined and dissolved in ethyl acetate (350 mL) to give a suspension that was warmed to 55-60° C. for 2 h. The suspension was filtered while hot and the resulting cake washed with ethyl acetate (2×150 mL) and hexanes (2×250 mL) to give 83.0 g (80.1%) of 4 as a white solid which was used in the next step without further purification. ¹H NMR (d₄-CH₃OH, 400 MHz) δ 3.60 (m, 1H), 3.46 (m, 1H), 3.29-3.32 (m, 2H), 2.72 (m, 1H), 2.09 (m, 1H), 1.70 (m, 1H), 1.45 (s, 9H).

Procedure B: A solution of the product of Example 3, Procedure B (4.35 kg), containing 2.13 kg (6.47 mol) of 3, in tetrahydrofuran (13.9 kg) was added to a stirred, cooled solution of potassium hydroxide (1.60 kg, 40.0 mol) in deionized water (2.00 kg) under a nitrogen atmosphere, while maintaining the temperature below 35° C. The reaction mixture was heated and maintained at 40-45° C. for 24 h, by which time GC and TLC analysis indicated that the reaction was complete. The mixture was cooled to 25° C. and washed with MTBE (34 kg), using 15 min of stirring and 15 min of settling time. The aqueous layer was collected and cooled to 1° C. A mixture of concentrated hydrochloric acid (2.61 kg, 26.5 mol) in deionized water (2.18 kg) was then added slowly, keeping the temperature of the mixture at <15° C. during and for 15 min after the addition. The pH of the solution was adjusted to 3.7 by further addition of hydrochloric acid. The white solid was collected by filtration, washed with water (16 kg), and vacuum dried at ambient temperature for 6 d. The dry solid weighed 1.04 kg. The filtrate was cooled to <10° C. and kept at that temperature as the pH was lowered by addition of more hydrochloric acid (1.6 L of 6 N was used; 9.6 mol; final pH=2). The white solid was collected by filtration, washed with water (8 L), and vacuum dried at 40° C. for 3 d. The dry solid weighed 0.25 kg. The combined solids (1.29 kg, 73% yield) were chromatographically identical to previously prepared samples.

### Synthesis of (R)-2-(1-(tert-butoxycarbonyl)pyrrolidine-3-yl)acetic acid (5)

Procedure A: A solution of (R)-2-(1-(tert-butoxycarbonyl)pyrrolidin-3-yl)malonic acid (83 g) in 1-methyl-2-pyrrolidinone (0.42 L) was stirred under nitrogen at 110-112° C. for 2 h. The reaction solution was sampled every hour and analyzed by HPLC to establish completion of the reaction. Upon completion of reaction the reaction solution was cooled to 20-25° C. The solution was mixed with de-ionized water (1.00 L), and MTBE (1.00 L) was added. The phases were separated, and the organic layer was collected. The aqueous phase was extracted with MTBE (1.00 L), then chloroform (1.00 L). The organic layers were combined and concentrated under reduced pressure at 50-55° C. to give an oil. This oil was dissolved in MTBE (2.00 L) and washed twice with 0.6N hydrochloric acid (2×1.00 L). The organic layer was collected and concentrated under reduced pressure at 50-55° C. to give a semi-solid. The semi-solid was suspended in 1:4 ethyl acetate/hexanes (100 mL), heated to 50° C., held for 30 min, cooled to −10° C., and filtered. The filtrate was concentrated under reduced pressure to give an oil, which was dissolved in MTBE (250 mL) and washed twice with 0.6N hydrochloric acid (2×100 mL). The organic layer was concentrated under reduced pressure at 50-55° C. to give a semi-solid which was suspended in 1:4 ethyl acetate/hexanes (50 mL), heated to 50° C., held for 30 min, cooled to −10° C., and filtered. The solids were collected, suspended in hexanes (200 mL), and collected by filtration to give 54.0 g (77.6%) of 5. ¹H NMR (CDCl₃, 400 MHz) δ 11.00 (br s, 1H), 3.63 (m, 1H), 3.45 (M, 1H), 3.30 (M, 1H), 2.97 (m, 1H), 2.58 (m, 1H), 2.44 (m, 2H), 2.09 (m, 1H), 1.59 (M, 1H), 1.46 (s, 9H).

Procedure B: A solution of (R)-2-(1-(tert-butoxycarbonyl)pyrrolidin-3-yl)malonic acid (1.04 kg, 3.81 mol) in 1-methyl-2-pyrrolidinone (6.49 kg) was stirred under nitrogen at 110° C. for 5 h, by which time TLC and HPLC analysis indicated that the reaction was complete. The reaction mixture was cooled to 25° C. (4 h) and combined with water (12.8 kg) and MTBE (9.44 kg). The mixture was stirred vigorously for 20 min, and the phases were allowed to separate (10 h). The organic phase was collected, and the aqueous phase was combined with MTBE (9.44 kg), stirred for 15 min, and allowed to settle (45 min). The organic phase was collected, and the aqueous phase was combined with MTBE (9.44 kg), stirred for 15 min, and allowed to settle (15 min). The three organic phases were combined and washed three times with 1N hydrochloric acid (8.44 kg portions) and once with water (6.39 kg), using 15 min of stirring and 15 min of settling time for each wash. The resulting solution was dried over anhydrous sodium sulfate (2.0 kg) and filtered. The filtrate was concentrated under reduced pressure at 31° C. (2 h) to give an solid. This solid was heated under vacuum for 4 h at 39° C. for 4 h and for 16 h at 25° C., leaving 704 g (81%) of 5 (99.7% purity by GC).

Procedure C (streamlined synthesis of 5, using 2 as starting material): A stirred mixture of sodium ethoxide in ethanol (21 weight percent, 343 g, 1.05 mol), ethanol (anhydrous, 300 mL) and diethyl malonate (168 g, 1.05 mol) was heated to 40° C. for 1.5 h. To this mixture was added a solution of (R)-tert-butyl 3-(methylsulfonyloxy)pyrrolidine-1-carboxylate (138 g, 0.592 mol) in ethanol (100 mL) and the reaction mixture was heated to 78° C. for 8 h. The cooled reaction mixture was diluted with water (2.0 L) and acidified to pH=3 with 6M HCl (100 mL). The aqueous ethanol mixture was extracted with toluene (1.0 L), and the organic phase concentrated under vacuum to afford 230 g of a red oil. The red oil was added at 85° C. to a 22.5 weight percent aqueous potassium hydroxide (748 g, 3.01 mol). After the addition was complete, the reaction temperature was allowed to slowly rise to 102° C. while a distillation of ethanol ensued. When the reaction temperature had reached 102° C., and distillation had subsided, heating was continued for an additional 90 min. The reaction mixture was cooled to ambient temperature and washed with toluene (2×400 mL). To the aqueous layer was added 600 mL 6M hydrochloric acid, while keeping the internal temperature below 20° C. This resulted in the formation of a precipitate, starting at pH of about 4-5. The suspension was filtered, and the filter cake was washed with 300 mL water. The solid was dried under vacuum to afford 77 g of (R)-2-(1-(tert-butoxycarbonyl)pyrrolidin-3-yl)malonic acid as an off-white solid (54% yield with respect to (R)-tert-butyl 3-(methylsulfonyloxy)pyrrolidine-1-carboxylate). ¹H NMR (DMSO-d₆, 400 MHz): δ 3.47 (m, 1H); 3.32 (m, 1H); 3.24 (m, 1H); 3.16 (m, 1H); 3.92 (m, 1H); 2.86 (m, 1H); 1.95 (m, 1H); 1.59 (m, 1H); 1.39 (s, 9H).

A suspension of (R)-2-(1-(tert-butoxycarbonyl)pyrrolidin-3-yl)malonic acid (15 g, 55 mmol) in toluene (150 mL) and dimethylsulfoxide (2 mL) was heated to reflux for a period of 2 h. The mixture was allowed to reach ambient and diluted with MTBE (150 mL). The organic solution was washed with 10% aqueous citric acid (2×200 mL), and the solvent was removed under vacuum to afford 11.6 g of (R)-2-(1-(tert-butoxycarbonyl)-pyrrolidin-3-yl)acetic acid as an off-white solid (92% yield). ¹H NMR (DMSO-d₆, 400 MHz): δ 12.1 (s, 1H); 3.36-3.48 (m, 1H); 3.20-3.34 (m, 1H); 3.05-3.19 (m, 1H, 2.72-2.84 (m, 1H); 2.30-2.42 (m, 1H), 2.22-2.30 (m, 2H); 1.85-2.00 (m, 1H); 1.38-1.54 (m, 1H), 1.35 (2, 9H).

### Synthesis of tert-butyl (R)-3-(2-hydroxyethyl)pyrrolidine-1-carboxylate (6)

Procedure A: A solution of (R)-2-(1-(tert-butoxycarbonyl)pyrrolidine-3-yl)acetic acid (49.0 g, 214 mmol) in tetrahydrofuran (THF) (200 mL) was cooled to −10° C. 250 mL (250 mmol) of a 1M borane in THF solution was added slowly to the flask while maintaining the temperature lower than 0° C. The solution was warmed to ambient temperature and stirred for 1 h. The solution was sampled hourly and analyzed by HPLC to establish completion of the reaction. Upon completion of the reaction, the solution was cooled to 0° C., and a 10% sodium hydroxide solution (80 mL) was added drop-wise over a 30 minute period to control gas evolution. The solution was extracted with 500 mL of a 1:1 hexanes/ethyl acetate solution. The organic layer was washed with saturated sodium chloride solution and dried with 10 g of silica gel. The silica gel was removed by filtration and washed with 100 mL of 1:1 hexanes/ethyl acetate. The organic layers were combined and concentrated under vacuum to give 6 (42 g, 91.3%) as a light-orange oil that solidified upon sitting. ¹H NMR (CDCl₃, 400 MHz) δ 3.67 (m, 2H), 3.38-3.62 (m, 2H), 3.25 (m, 1H), 2.90 (m, 1H), 2.25 (m, 1H), 1.98-2.05 (m, 1H), 1.61-1.69 (m, 2H), 1.48-1.59 (m, 2H), 1.46 (s, 9H).

Procedure B: Borane-THF complex (3.90 kg or L of 1M in THF, mol) was added slowly to a stirred solution of (R)-2-(1-(tert-butoxycarbonyl)pyrrolidine-3-yl)acetic acid (683 g, 3.03 mol) in THF (2.5 kg), kept under nitrogen gas, and using a water bath to keep the temperature between 23 and 28° C. The addition took 1.75 h. Stirring at 25° C. was continued for 1 h, after which time GC analysis indicated complete reaction. The reaction mixture was cooled to <10° C. and maintained below 25° C. as 10% aqueous sodium hydroxide (1.22 kg) was slowly added. The addition took 40 min. The mixture was stirred 1 h at 25° C., and then combined with 1:1 (v/v) heptane/ethyl acetate (7 L). The mixture was stirred for 15 min and allowed to separate into phases (1 h). The organic phase was withdrawn, and the aqueous phase was combined with a second 7 L portion of 1:1 heptane/ethyl acetate. This was stirred for 15 min and allowed to separate into phases (20 min). The organic phase was again withdrawn, and the combined organic phases were washed with saturate aqueous sodium chloride (4.16 kg), using 15 min of mixing and 1 h of settling time. The organic phase was combined with silica gel (140 g) and stirred 1 h. The anhydrous sodium sulfate (700 g) was added, and the mixture was stirred for 1.5 h. The mixture was filtered, and the filter cake was washed with 1:1 heptane/ethyl acetate (2 L). The filtrate was concentrated under vacuum at <40° C. for 6 h. The resulting oil weighed 670 g (103% yield) and contains traces of heptane, but is otherwise identical to previously prepared samples of 6, by NMR analysis.

### Synthesis of tert-butyl (R)-3-(2-(methylsulfonyloxy)ethyl)pyrrolidine-1-carboxylate (7)

Procedure A: To a solution of tert-butyl (R)-3-(2-hydroxymethyl)pyrrolidine-1-carboxylate (41.0 g, 190 mmol)) was added triethylamine (40 mL) in toluene (380 mL) and cooled to −10° C. Methanesulfonyl chloride (20.0 mL, 256 mmol) was added slowly so as to maintain the temperature around −5 to 0° C. The solution was warmed to ambient temperature and stirred for 1 h. The solution was sampled hourly and analyzed by HPLC to establish completion of the reaction. Upon completion of reaction, the solution was filtered, and the filtrate was washed with a 5% sodium bicarbonate solution (250 mL). The organic layer was collected and washed with a saturated aqueous sodium chloride solution (250 mL). The organic layer was collected, dried over silica gel (10 g), and concentrated under vacuum to give 7 (53.0 g, 92.8%) as a light-yellow viscous oil. ¹H NMR (CDCl₃, 400 MHz) δ 4.26 (t, J=6.8 Hz, 2H), 3.41-3.63 (m, 2H), 3.27 (m, 1H), 3.02 (s, 3H), 2.92 (m, 1H), 2.28 (m, 1H), 2.05 (m, 1H), 1.83 (m, 2H), 1.50-1.63 (m, 1H), 1.46 (s, 9H).

Procedure B: Under a nitrogen atmosphere, a solution of triethylamine (460 g, 4.55 mol) and tert-butyl (R)-3-(2-hydroxymethyl)pyrrolidine-1-carboxylate (the entire sample from Example 7, Procedure B, 3.03 mol) in toluene (5.20 kg) was stirred and cooled to 5° C. Methanesulfonyl chloride (470 g, 4.10 mol) was added slowly, over a 1.25 h, keeping the temperature below 15° C. using ice bath cooling. The mixture was gradually warmed (over 1.5 h) to 35° C., and this temperature was maintained for 1.25 h, at which point GC analysis indicated that the reaction was complete. The mixture was cooled to 25° C., and solids were filtered off and the filter cake washed with toluene (1.28 kg). The filtrate was stirred with 10% aqueous sodium bicarbonate (4.0 kg) for 15 min, and the phases were allowed to separate for 30 min. The organic phase was then stirred with saturated aqueous sodium chloride (3.9 kg) for 30 min, and the phases were allowed to separate for 20 min. The organic phase was combined with silica gel (160 g) and stirred for 1 h. Anhydrous sodium sulfate (540 g) was added, and the mixture was stirred an additional 40 min. The mixture was then filtered, and the filter cake was washed with toluene (460 g). The filtrate was concentrated under vacuum at 50° C. for 5 h, and the resulting oil was kept under vacuum at 23° C. for an additional 8 h. This left 798 g of 7, 93% pure by GC analysis.

### Synthesis of tert-butyl (R)-3-vinylpyrrolidine-1-carboxylate (9)

Procedure A: A solution of tert-butyl (R)-3-((methylsulfonyloxy)ethyl)pyrrolidine-1-carboxylate (49.0 g, 167 mmol), sodium iodide (30.0 g, 200 mmol) and 1,2-dimethoxyethane (450 mL) was stirred at 50-60° C. for 4 h. The solution was sampled hourly and analyzed by HPLC to establish completion of the reaction. Upon completion of reaction, the solution was cooled to −10° C., and solid potassium tert-butoxide (32.0 g, 288 mmol) was added while maintaining temperature below 0° C. The reaction mixture was warmed to ambient temperature and stirred for 1 h. The mixture was sampled hourly and analyzed by HPLC to establish completion of the reaction. Upon completion of reaction, the mixture was filtered through a pad of diatomaceous earth (25 g dry basis). The cake was washed with 1,2-dimethoxyethane (100 mL). The combined filtrates were concentrated under vacuum, to yield an orange oil with suspended solids. The oil was dissolved in hexanes (400 mL), stirred for 30 min, and filtered to remove the solids. The organic layer was dried over silica gel (10 g), and concentrated under vacuum to give 9 (26.4 g, 82.9%) as a colorless oil. ¹H NMR (CDCl₃, 400 MHz) δ 5.77 (m, 1H), 5.10 (dd, J=1.2 Hz, J=16 Hz, 1H), 5.03 (dd, J=1.2 Hz, J=8.8 Hz, 1H), 3.41-3.59 (m, 2H), 3.29 (m, 1H), 3.05 (m, 1H), 2.78 (m, 1H), 2.01 (m, 1H), 1.62-1.73 (m, 1H), 1.46 (m, 9H).

Procedure B: A solution of tert-butyl (R)-3-(2-(methylsulfonyloxy)ethyl)pyrrolidine-1-carboxylate (792 g of the product of Example 7, Procedure B, −2.5 mol), sodium iodide (484 g, 3.27 mol) and 1,2-dimethoxyethane (7.2 L) was stirred at 55° C. for 4.5 h under nitrogen, at which time GC analysis indicated that the reaction was complete. The solution was cooled to <10° C., and solid potassium tert-butoxide (484 g, 4.32 mol) was added in portions (1.25 h addition time) while maintaining temperature below 15° C. The reaction mixture was stirred 1 h at 5° C., warmed slowly (6 h) to 20° C., and stirred at 20° C. for 1 h. The solution was filtered through a pad of diatomaceous earth (400 g dry basis). The filter cake was washed with 1,2-dimethoxyethane (1.6 kg). The combined filtrates were concentrated under vacuum, and the semisolid residue was stirred with heptane (6.0 L) for 2 h. The solids were removed by filtration (the filter cake was washed with 440 mL of heptane), and the filtrate was concentrated under vacuum at 20° C. to give 455 g of 9 (90.7% pure). A sample of this material (350 g) was fractionally distilled at 20-23 torr to give 296 g of purified 9 (bp 130-133° C.) (>99% pure by GC analysis).

### Synthesis of (R)-5-((E)-2-pyrrolidin-3-ylvinyl)pyrimidine (11)

Nitrogen was bubbled through a solution of (R)-tert-butyl 3-vinylpyrrolidine-1-carboxylate (25 g, 127 mmol), 5-bromopyrimidine (30.3 g, 190 mmol), 1,1′-bis(diphenylphosphino)ferrocene (2.11 g, 3.8 mmol), and sodium acetate (18.8 gr, 229 mmol) in N,N-dimethylacetamide (250 mL) for 1 h, and palladium acetate (850 mg, 3.8 mmol) was added. The reaction mixture was heated to 150° C. at a rate of 40° C./h and stirred for 16 h. The mixture was cooled to 10° C. and quenched with water (750 mL) while maintaining an internal temperature below 20° C. MTBE (300 mL) was added, followed by diatomaceous earth (40 g, dry basis). The suspension was stirred for 1 h at ambient temperature and filtered through a bed of diatomaceous earth. The residue was washed with MTBE (2×100 mL) and the filtrate was transferred to a 2-L vessel equipped with an overhead stirrer and charged with activated charcoal (40 g). The suspension was stirred for 2 h at ambient temperature and filtered through diatomaceous earth. The residue was washed with MTBE (2×100 mL), and the filtrate was concentrated in vacuo to afford 28.6 g of an orange oil. The oil is dissolved in MTBE (100 mL) and Si-Thiol® (2.0 g, 1.46 mmol thiol/g, Silicycle Inc.) was added. The suspension was stirred under nitrogen at ambient temperature for 3 h, filtered through a fine filter, and held in a glass container.

To a solution of 6 M HCl (70 mL) was added the filtrate over a period of 30 min while maintaining the internal temperature between 20° C. and 23° C. The mixture was stirred vigorously for 1 h and the organic layer removed. The remaining aqueous layer was basified with 45 wt % KOH (50 mL), and the resulting suspension was extracted once with chloroform (300 mL). Evaporation of the solvent in vacuo (bath temperature at 45° C.) gave 16.0 g (71.8%), of (R)-5-((E)-2-pyrrolidin-3-ylvinyl)pyrimidine free base as a red oil, which is immediately dissolved in isopropanol (50 mL) and used for salt formation.

### Salts

(R)-5-((E)-2-Pyrrolidin-3-ylvinyl)pyrimidine free base (10 mg, 0.057 mmol) was dissolved in either isopropyl acetate or acetonitrile. The solutions were treated with 0.5 (hemi) to 1.1 (mono) eq. of a corresponding acid, warmed to 50° C., and cooled slowly to ambient temperature overnight. The solvent was then evaporated under vacuum without heating.

### Example 2

### In Vivo Methods: L-Dopa-Induced Dyskinesia

The subtype selective NNR agonist Compound A, stimulates α41β2* and to a lesser extent α6* NNRs. Toxicological studies show that there is a low incidence of peripheral side effects in rodents, dogs, and primates and a reasonable therapeutic index, consistent with its interaction primarily at α4β2* and α6* nicotinic receptors. In safety studies in rats, Compound A, which is coded as PD1 for these examples, was not associated with any significant detrimental effects using acute free base oral doses up to 30 mg/kg. There was no behavioral sensitization, and Compound A (PD1) did not increase locomotor activity in rats, suggesting low liability for abuse. Experiments were performed using male Sprague-Dawley rats that were unilaterally lesioned with 6-OHDA (Sigma Chemical Co., St. Louis, Mo.) as previously described (Cenci et al., 1998; Cenci et al., 2002). 6-OHDA was dissolved in 0.02% ascorbic acid/saline at a concentration of 3 μg/μl. Compound A was systemically infused via osmotic minipumps (Alzet model 2006; Durect Corporation, Cupertino, Calif., USA), which delivered drug at a rate of 3.6 μl/day for 6 wk. This route was selected because nicotine most effectively reduced L-dopa-induced AIMs in rats when given continuously via this route (Bordia, T., Campos, C., Huang, L. Z. & Quik, M., “Continuous And Intermittent Nicotine Treatment Reduces L-3,4-Dihydroxyphenylalanine (L-Dopa)-Induced Dyskinesias In A Rat Model Of Parkinson's Disease,” J Pharmacol Exp Ther 327: 239-47 (2008)). Pumps were pre-filled with either sterilized water or Compound A base in water (pH 7.0) to provide the required doses (0.03, 0.1, 0.3 mg/kg/d). The minipumps were subcutaneously implanted along the dorsal aspect of the neck between the shoulder blades, according to the manufacturer's instruction, under isofluorane anesthesia. After implantation, rats were administered buprenorphine (0.02 mg/kg sc) for post-operative pain. Two weeks after initiation of drug treatment, the rats were administered single daily subcutaneous injections of 6 to 8 mg/kg L-dopa methyl ester plus 15 mg/kg benserazide. Two μl aliquots were stereotaxically injected at each of two sites for a total of 8 to 12 μg into the right ascending dopamine fiber bundle. Infusion of 6-OHDA into the target area was over a 2-min period, with the cannula maintained at the site of injection for a further 2 min. Two to three weeks after lesioning, rats were tested for drug-induced rotational behavior in an automated behavioral measurement apparatus (ROTOMAX, AccuScan Instruments Inc. Columbus, Ohio, USA). Each rat was placed in a cylindrical glass chamber for 30 min for acclimatization, after which 4.0 mg/kg amphetamine (Sigma Chemical Co., St. Louis, Mo.) was administered intraperitoneally (ip). Amphetamine-induced rotational behavior was monitored for 90 min, with rats making at least 200 ipsilateral turns/90 min used for further study.

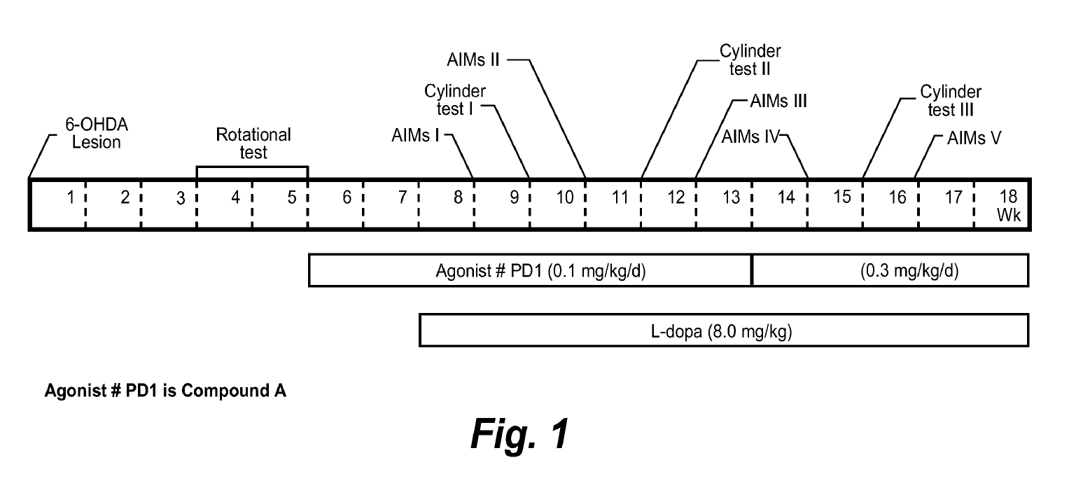

L-dopa-induced AIMs were determined as indicated. See FIG. 1 for experiment dosing and testing regimen. Behavioral testing was done in the morning starting between 9 and 10 AM. Three different AIMs subtypes were measured including (1) axial dystonia, contralateral twisted posturing of the neck and upper body; (2) abnormal orolingual movements, stereotyped jaw movements and contralateral tongue protrusion; and (3) abnormal forelimb movements, repetitive rhythmic jerks or dystonic posturing of the contralateral forelimb and/or grabbing movements of the contralateral paw. See, for example, Carta M, Lindgren HS, Lundblad M, Stancampiano R, Fadda F, Cenci M A (2006), Role of Striatal L-DOPA in the Production of Dyskinesia in 6-hydroxydopamine Lesioned Rats. J Neurochem 96:1718-172; Cenci M A, Lee C S, Bjorklund A (1998) L-DOPA-induced dyskinesia in the rat is associated with striatal overexpression of prodynorphin- and glutamic acid decarboxylase mRNA; Eur J Neurosci 10:2694-2706; Cenci M A, Whishaw I Q, Schallert T (2002) Animal models of neurological deficits: how relevant is the rat? Nat Rev Neurosci 3:574-579; Bordia, T., Campos, C., Huang, L. Z. & Quik, M., “Continuous And Intermittent Nicotine Treatment Reduces L-3,4-Dihydroxyphenylalanine (L-Dopa)-Induced Dyskinesias In A Rat Model Of Parkinson's Disease,” J Pharmacol Exp Ther 327: 239-47 (2008)). Rats were scored on a scale from 0 to 4 for each of these three AIM subtypes as follows: 1=occasional; 2=frequent; 3=continuous but interrupted by sensory distraction; and 4=continuous, severe, not interrupted by sensory distraction. Animals were evaluated for AIMs for an initial 20 min baseline period, as well as for 3 h following L-dopa injection by a blinded rater. Assessment of the different subtypes of AIMs was done over 20 min sessions, thus yielding a total of 9 sessions of testing per animals. The maximum possible score for each animal was thus 108 (maximum score per session=12; number of sessions over 3 h=9).

All statistical analyses were performed using GraphPad Prism® (GraphPad Software, Inc, San Diego, Calif.). Values are the mean±SEM of the indicated number of rats, and represent data from one to two separate testing periods. Differences in rating scores between groups were analyzed using nonparametric tests (Mann-Whitney test). For the time course studies, repeated measures analysis of variance (ANOVA) followed by Bonferroni multiple comparison test was used. A level of 0.05 was considered significant.

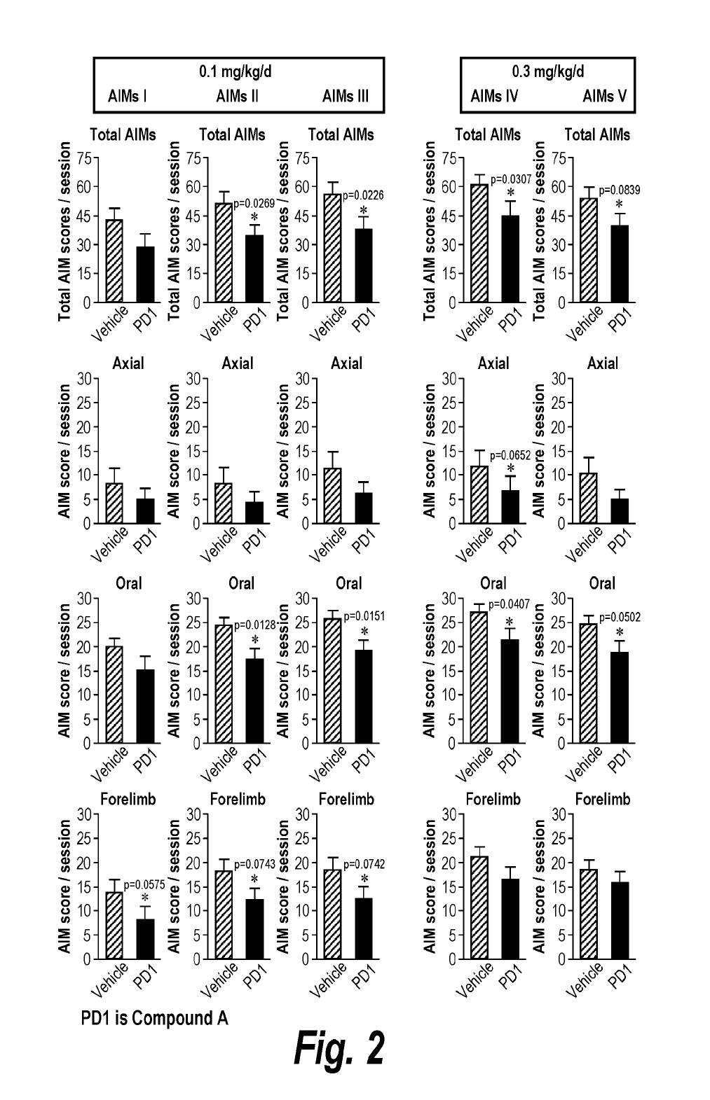

Interestingly, Compound A (PD1) at doses of 0.1 and 0.3 mg/kg/day resulted in a significant reduction in total or some subset (axial, oral and/or forelimb) of L-dopa-induced AIMs (See FIG. 2) across multiple assessments. This included a significant decline in total AIMs during the second and third assessments (with 0.1 mg/kg/day) and during the fourth assessment (with 0.3 mg/kg/day), with trends for declines in total AIMs during the first and fifth AIMs assessments. Importantly, Compound A (PD1) resulted in no detectable adverse effects (i.e., on body weight, body temperature, grooming, urination, defecation, secretion).

### Example 3

### Motor Function

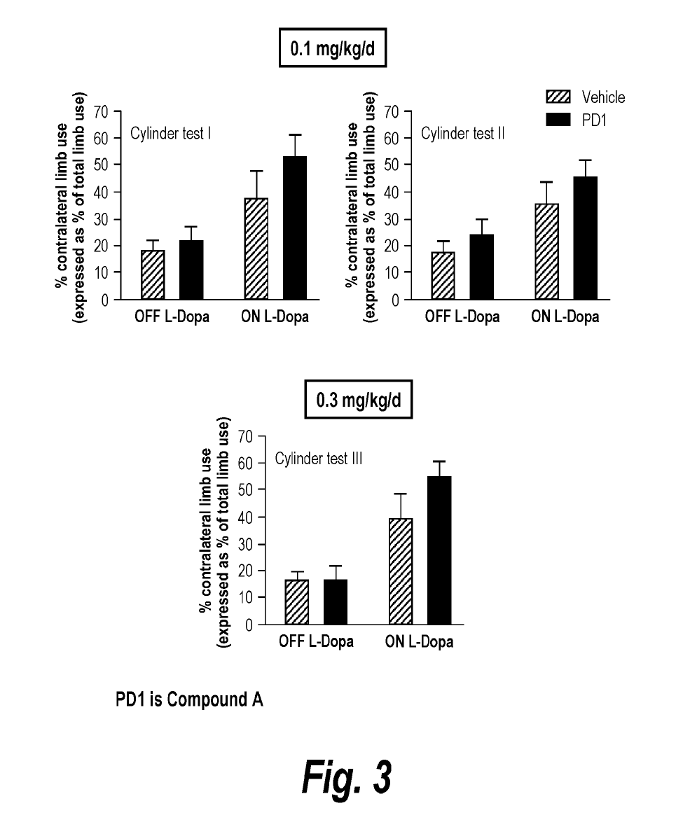

Because drugs that reduce AIMs may worsen Parkinsonism, the effect of Compound A (PD1) was also tested on motor function in Parkinsonian rats (see FIG. 3) using the limb use asymmetry or cylinder test. The cylinder test was used as an index of behavioral function after unilateral nigrostriatal damage, with explorative activity analyzed as previously described. Animals were placed in a transparent cylinder (20 cm diameter×30 cm height) or in a transparent cage and evaluated over a 5 min period. A mirror was placed behind the cylinder/cage to allow the raters to view forelimb movements when the rat was turned away from the rater. Wall exploration was expressed in terms of the percentage of use of the impaired forelimb (contralateral to the lesion) compared to the total number of limb use movements. Two raters, one blinded to the treatment status of the rats, performed the ratings. Correlation analyses yielded a high inter-rater reliability (R=0.99). This test also offers the advantage that it does not require the use of dopaminergic drugs (i.e., amphetamine) to detect abnormalities. The rat is placed in a transparent cylinder, which results in vertical exploration and the percentage of contact of the unimpaired and impaired forelimb against the cylinder evaluated (Cenci, M. A. and M. Lundblad, “Post-Versus Presynaptic Plasticity In L-Dopa-lnduced Dyskinesia,”. J Neurochem, 99: 381-392 (2006); Tillerson, J. L. et al., “Forced Limb-Use Effects On The Behavioral And Neurochemical Effects Of 6-Hydroxydopamine,” J Neurosci 21, 4427-35 (2001); and Meredith, G. E. & U. J. Kang, “Behavioral Models Of Parkinson's Disease In Rodents: A New Look At An Old Problem,” Mov Disord, 21: 1595-606 (2006).

The results show that Compound A (PD1) does not worsen baseline motor function (off L-dopa) nor does it worsen the enhanced limb use with L-dopa treatment at either dose tested across the course of the experiment. In fact, there was a small trend for improvement. Overall, Compound A (PD1) appears to be a promising candidate to reduce L-dopa-induced AIMs without worsening Parkinsonism (See FIG. 3).

Unless otherwise noted, Compound A, PD1, (R)-5-((E)-2-pyrrolidin-3-ylvinyl)pyrimidine, was dosed as its hemi-galactarate salt, however dose was calculated as the free-base equivalent.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A method for treating L-dopa-induced dyskinesia or abnormal involuntary movements comprising by administering one or more compound, which targets one or more of α4β2*, α6β2*, or α7 NNRs.

2. A method for reducing L-dopa-induced dyskinesia or abnormal involuntary movements comprising administering one or more compound, which targets one or more of α4β2*, α6β2*, or α7 NNRs.

3. A method for delaying the onset or progression of L-dopa-induced dyskinesia or abnormal involuntary movements comprising administering one or more compound, which targets one or more of α4β2*, α6β2*, or α7 NNRs.

4. A method for improving Parkinsonism comprising administering one or more compound, which targets one or more of α4β2*, α6β2*, or α7 NNRs.

5. A method for treating a disease responsive to L-dopa comprising administering L-dopa and one or more compound, which targets one or more of α4β2*, α6β2*, or α7 NNRs.

6. The method of claim 5, wherein the disease responsive to L-dopa is Parkinson's disease.

7. A method for treating, reducing, or delaying progression of L-dopa-induced dyskinesia or abnormal involuntary movements by administering a compound which targets both α4β2* and α6β2* NNRs.

8. A method for treating, reducing, or delaying progression of L-dopa-induced dyskinesia or abnormal involuntary movements by administering a compound which targets α7 NNRs.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A combination comprising:
a. L-dopa; and
b. one or more compound, which targets one or more of α4β2*, α6β2*, or α7 NNRs.

25. A treatment regimen comprising:
a. L-dopa; and
b. one or more compound, which targets one or more of α4β2*, α6β2*, or α7 NNRs,
wherein the one or more compound, which targets one or more of α4β2*, α6β2*, or α7 NNRs, is administered concurrently with the L-dopa.
